(19) United States
(12) Patent Application Publication
(10) Pub. No.: US 2022/0105013 A1
(43) Pub. Date: Apr. 7, 2022

(54) EYELASH ADHESIVE EYELINER FORMULATION

(71) Applicant: Taylored Lashes, LLC, Moulton, TX (US)

(72) Inventor: Taylor Anne Nichols, College Station, TX (US)

(73) Assignee: Taylored Lashes, LLC, Moulton, TX (US)

(21) Appl. No.: 17/060,311

(22) Filed: Oct. 1, 2020

(51) Int. Cl. A61K 8/34; A61K 8/81; A61K 8/60; A61K 8/19; A41G 5/02

(52) U.S. Cl. CPC A61K 8/345; A61K 8/8129; A61K 8/8117; A61K 2800/42; A61K 8/34; A61K 8/19; A41G 5/02; A61K 8/60

(57) ABSTRACT

A lash adhesive liner formulation includes a pressure-sensitive adhesive and between about 0.02 and about 0.3 weight percent glycerin, where the pressure-sensitive adhesive is activated by pressing a lash band of a lash against the pressure-sensitive adhesive that has been applied to the eyelid. The lash is secured near a lashline of an eyelid by applying a layer of lash adhesive liner formulation to the eyelid, allowing the lash adhesive liner formulation that has been applied to the eyelid to dry for 5-15 seconds so that the lash adhesive liner formulation becomes tacky, and then pressing the lash band of the lash firmly against the tacky lash adhesive liner. The lash adhesive liner formulation may include a colorant, such that the formulation may also substitute for an eyeliner.

### BACKGROUND

The present disclosure relates to a cosmetic product, such as an adhesive formulation used to secure a false eyelashes to an eyelid.

### BACKGROUND OF THE RELATED ART

When you meet a person, one of the first things that you notice are their eyes. Eyes may convey significant social and emotional meaning in many contexts and a person that makes appropriate eye contact may be viewed as being confident and caring. It is no wonder that people want their eyes to be attractive to others.

Cosmetics are available to enhance the appearance of a person's eyes. For example, eyeliner may be used to define the contour of the eyes. Mascara may be applied to eyelashes to further enhance the eyes and provide greater definition and interest. Furthermore, if the person's own eyelashes are not naturally long or full, false eyelashes may be applied along the edge of the eyelid to give the appearance of naturally long and full eyelashes. These and other cosmetics may be used to subtly or dramatically enhance or change the overall appearance of the eyes.

False eyelashes (or simply “lashes) may be made with human hair, animal fur, or synthetic materials. For example, lashes may be made with animal fur, such as mink, sable and fox. Synthetic lashes may be made with acrylic resin or polybutylene terephthalate (PBT). Polybutylene terephthalate is a thermoplastic polymer that may be heated and then molded into the desired lash shape to make a strong and naturally glossy filament. Regardless of the source, the individual hairs or filaments are secured to a lash band or strip to make a lash. A person may cut the lash band to a desired length to fit their eyelid or to achieve a particular look. After trimming a lash to a desired length, a small brush, tube or other means may be used to carefully apply eyelash glue to an outer seam of the lash band. The lash band is then carefully placed on the eyelid as close as possible to the natural lashline.

### BRIEF SUMMARY

Some embodiment provide a lash adhesive liner formulation including a pressure-sensitive adhesive and between about 0.02 and about 0.3 weight percent glycerin, where the pressure-sensitive adhesive is activated by pressing a lash band against the pressure-sensitive adhesive that has been applied to the eyelid.

Some embodiments provide a lash adhesive liner formulation including about 35 weight percent styrene acrylate copolymer, about 50 weight percent water, about 10 weight percent 1,2-propanediol, about 2 weight percent polyvinyl alcohol, about 2 weight percent polysorbate 80, about 0.27 weight percent phenoxyethanol, about 0.21 weight percent octanediol, and about 0.12 weight percent ethylhexylglycerin.

Some embodiments provide a lash adhesive liner formulation including between about 13 and about 45 weight percent styrene acrylate copolymer, about 32 weight percent water, about 7.3 weight percent 1,2-propanediol, about 1 weight percent polyvinyl alcohol, about 0.9 weight percent polysorbate 80, about 0.27 weight percent phenoxyethanol, about 0.21 weight percent octanediol, about 0.12 weight percent ethylhexylglycerin, and one or more colorant composition. In one option, the styrene acrylate copolymer has a concentration of about 13 weight percent, wherein the one or more colorant includes about 45 weight percent carbon black. In another option, the styrene acrylate copolymer has a concentration of about 45 weight percent, wherein the one or more colorant includes about 1.2 weight percent triiron tetraoxide, about 6 weight percent diiron trioxide, and about 6 weight percent iron oxide.

Some embodiments provide a method of securing a lash to a lashline of an eyelid. The method may include applying a layer of lash adhesive liner formulation to an eyelid, wherein the lash adhesive liner formulation comprises a pressure-sensitive adhesive and greater than 0.2 weight percent glycerin, then allowing the lash adhesive liner formulation that has been applied to the eyelid to dry for 5-15 seconds so that the lash adhesive liner formulation becomes tacky, and then pressing a lash band of a lash firmly against the tacky lash adhesive liner formulation.

### BRIEF DESCRIPTION OF THE SEVERAL VIEWS OF THE DRAWINGS

FIG. 1 is a diagram of a person's eyes, eyelids, and natural eyelashes.

FIG. 2 is a diagram of a pair of lashes.

FIG. 3 is a diagram of pen for applying a lash adhesive liner formulation.

FIG. 4 is a diagram of a pen being used to apply a lash adhesive liner formulation on the eyelid of the person's left eye adjacent the eyelashes.

FIG. 5 is a diagram of the same left eye shown in FIG. 4 after pressing a band of the left lash over the lash adhesive liner formulation applied in FIG. 4.

FIG. 6 is a diagram of the person's eyes after securing the left and right lashes to the left and right eyes, respectively.

### DETAILED DESCRIPTION

Some embodiment provide a lash adhesive eyeliner formulation (or simply “lash adhesive liner formulation”) including a pressure-sensitive adhesive and between about 0.02 and about 0.3 weight percent glycerin, where the pressure-sensitive adhesive is activated by pressing a lash band against the pressure-sensitive adhesive that has been applied to the eyelid. For example, the glycerin concentration may be between about 0.12 and about 0.3 weight percent. The amount of glycerin in the formulation is preferably effective to provide skin moisturization and improve the release of the lash adhesive liner formulation from the lash band and the skin without causing the lash adhesive liner formulation to be slick to the touch. A non-limiting example of the glycerin is ethylhexylglycerin.

The lash adhesive liner formulation includes a pressure-sensitive adhesive that is activated by pressing a lash strip against the pressure-sensitive adhesive that has been applied to the eyelid. Pressure-sensitive adhesives are a non-reactive type of adhesive that forms a bond when light pressure is applied to the adhesive. Accordingly, the pressure-sensitive lash adhesive liner formulation enables false eyelashes (herein after referred to as “lashes”) to be both removable and re-stickable. For example, lashes that have been applied incorrectly, or in an unintended position, can be easily removed by pulling the lashes from outer end of the band. Then, since the pressure-sensitive adhesive liner formulation remains on the eyelid, the lashes maybe re-applied (i.e., the lashes are “re-stickable”) to the original application of the lash adhesive liner formulation by pressing the lash band against the lash adhesive liner formulation. Accordingly, a user may get multiple attempts at positioning a given lash without needing to reapply the lash adhesive liner formulation. However, despite the forgiving nature of the lash adhesive liner formulation, the lash adhesive liner formulation may also provide an all-day hold of the lashes.

Whereas a structural adhesive forms a hardened permanent bond through a process such as solvent evaporation, reaction with ultraviolet (UV) radiation, chemical reaction, or cooling, a pressure-sensitive adhesive (PSA) forms a bond simply by the application of light pressure against the adhesive. Pressure-sensitive adhesives are designed with a balance between flow and resistance to flow. The bond forms because the adhesive is soft enough to flow, yet the bond has strength because the adhesive is hard enough to resist flow when stress is applied to the bond. Pressure-sensitive adhesives may exhibit viscoelastic properties (i.e., both viscous and elastic properties) which are used for proper bonding. In contrast with structural adhesives, whose strength is evaluated as lap shear strength, pressure-sensitive adhesives are characterized by their shear and peel resistance as well as their initial tack. These properties may be dependent upon the components of the formulation, coating thickness, temperature, and other parameters.

In some embodiments, the lash adhesive liner formulation may further include one or more colorant compound that establishes a color of the lash adhesive liner formulation. The lash adhesive liner formulation may include any one or more colorant compound to establish lash adhesive liner formulations having a wide range of colors including, without limitation, black and brown. As a result of the inclusion of one or more colorant compound, the lash adhesive liner formulation may serve as a substitute for eyeliner. Therefore, the lash adhesive liner formulation may be applied to the eyelid in the same manner as eyeliner. For example, a person may apply the lash adhesive liner formulation over an area of the eyelid where the person desires to have the appearance of eyeliner and may subsequently press a lash band of a desired lash against a portion of the applied formulation that is closest to the eyelashes. In alternative embodiments, the lash adhesive liner formulation may be formulated with only clear ingredients and without any colorant or opaque ingredients, such that the lash adhesive liner formulation is clear.

In some embodiments, the lash adhesive liner formulation may be characterized in that two to three layers of the lash adhesive liner formulation may be applied to the skin of an eyelid with an eyeliner pen to provide a coating having sufficient thickness to secure a lash band that is pressed against the layers. The lash adhesive liner formulation may be characterized in that the lash adhesive liner formulation becomes tacky in about 5 to 15 seconds after being applying to an eyelid.

When a person applies a lash to an eyelid, the pressure of the lash band against the lash adhesive liner formulation activates the pressure-sensitive adhesive to form a bond between the adhesive and the lash band. The pressing of the lash band against the pressure-sensitive adhesive also has the effect of pressing the pressure-sensitive adhesive against the skin of the eyelid such that the portion of the pressure-sensitive that is in contact with the skin is similarly activated to form a bond with the skin of the eyelid.

In some embodiments, the bond between the pressure-sensitive adhesive and the lash will secure the lash to the eyelid until the person intentionally removes the lash. Accordingly, the person may wear a pair of the lashes for a day or longer. However, if the person does not achieve a desired placement of the lashes on the first or subsequent attempt, the lash adhesive liner formulation allows the person to remove and reapply the lash any number of times. In other words, the pressure-sensitive adhesive enables a lash band to be both removable after being applied to the pressure-sensitive adhesive and re-stickable after being removed from the pressure-sensitive adhesive. When the lash band is removed from an eyelid, the lash adhesive liner formulation preferably remains on the eyelid. So, embodiments of the lash adhesive liner formulation make it possible to remove a first lash and apply a second lash to the originally applied lash adhesive liner formulation without reapplying the lash adhesive liner formulation. This may be particularly beneficial where the person selects and applies a first lash to the eyelid and, upon subsequent inspection in a mirror, decides that a second lash would be a better selection for their desired appearance. Furthermore, a person may decide to wear a second lash on the eyelid (i.e., “double lashes”) to achieve an even thicker appearance, such that the lash band of the second lash may be applied to the eyelid adjacent the lash band of the first lash. If the lash adhesive liner was applied over a sufficiently wide area along the lash line, then the lash band of the second lash may be pressed into position adjacent the lash band of the first lash without applying any additional adhesive.

In some embodiments, the pressure-sensitive adhesive may include from 1 to 2 weight percent polyvinyl alcohol. Such embodiments may further include from 13 to 45 weight percent styrene acrylate copolymer.

Some embodiments provide a lash adhesive liner formulation including about 35 weight percent styrene acrylate copolymer (such as CAS No. 9010-92-8), about 50 weight percent water (such as CAS No. 7732-18-5), about 10 weight percent 1,2-propanediol (such as CAS No. 57-55-6), about 2 weight percent polyvinyl alcohol (such as CAS No. 25213-24-5), about 2 weight percent polysorbate 80 (such as CAS No. 9005-65-6), about 0.27 weight percent phenoxyethanol (such as CAS No. 122-99-6), about 0.21 weight percent octanediol (such as CAS No. 1117-86-8), and about 0.12 weight percent ethylhexylglycerin (such as CAS No. 70445-33-9). This lash adhesive liner formulation may be prepared without any colorant, such that the lash adhesive liner formulation is clear or translucent. The lash adhesive liner formulation of this embodiment may incorporate other components, features, uses and/or advantages of any other embodiment described herein without limitation.

The foregoing and subsequent embodiments identify various components of the lash adhesive liner formulation using the International Nomenclature Cosmetic Ingredient (INCI) names, which are systematic names that are internationally recognized to identify cosmetic ingredients. These names are developed by the International Nomenclature Committee (INC) and published by the Personal Care Products Council (PCPC) in the International Cosmetic Ingredient Dictionary and Handbook, available electronically as wINCI. In some embodiments, components of the lash adhesive liner formulation may be further identified by a Chemical Abstract Service (CAS) registry number, which is a unique numerical identifier assigned by the Chemical Abstracts Service (CAS) to every chemical substance described in the open scientific literature. In some instances, a component, such as a colorant or pigment, may be identified by an associated Cosmetic Ingredient (CI) number as established by INCI.

Some embodiments provide a lash adhesive liner formulation including between about 13 and about 45 weight percent styrene acrylate copolymer (such as CAS No. 9010-92-8), about 32 weight percent water (such as CAS No. 7732-18-5), about 7.3 weight percent 1,2-propanediol (such as CAS No. 57-55-6), about 1 weight percent polyvinyl alcohol (such as CAS No. 25213-24-5), about 0.9 weight percent polysorbate 80 (such as CAS No. 9005-65-6), about 0.27 weight percent phenoxyethanol (such as CAS No. 122-99-6), about 0.21 weight percent octanediol (such as CAS No. 1117-86-8), about 0.12 weight percent ethylhexylglycerin (such as CAS No. 70445-33-9), and one or more colorant composition. In one option, the styrene acrylate copolymer has a concentration of about 13 weight percent, wherein the one or more colorant includes about 45 weight percent carbon black (CI 77266) to form a lash adhesive liner formulation with a black color. In another option, the styrene acrylate copolymer has a concentration of about 45 weight percent, wherein the one or more colorant includes about 1.2 weight percent triiron tetraoxide (CI 77499), about 6 weight percent diiron trioxide (CI 77491), and about 6 weight percent iron oxide (CI 77492) to form a lash adhesive liner formulation with a brown color. The lash adhesive liner formulation of this embodiment may incorporate other components, features, uses and/or advantages of any other embodiment described herein without limitation.

Some embodiments provide a method of securing a lash to a lashline of an eyelid. The method may include applying a layer of a lash adhesive liner formulation to an eyelid, wherein the lash adhesive liner formulation comprises a pressure-sensitive adhesive and greater than 0.2 weight percent glycerin, then allowing the layer of the lash adhesive liner formulation that has been applied to the eyelid to dry for 5-15 seconds so that the layer of the lash adhesive liner formulation becomes tacky, and then pressing a lash band of a lash firmly against the tacky lash adhesive liner formulation. In one option, the lash adhesive liner formulation may be applied to the eyelid using an eyeliner pen. In another option, the step of applying a layer of the lash adhesive liner formulation to an eyelid may include applying multiple layers, such as two to three layers, of the lash adhesive liner formulation to the eyelid.

In some embodiments, the method may further include applying an additional amount of the lash adhesive eyeliner to the lash band, and then allowing the lash adhesive liner formulation that has been applied to the lash band to dry for about 5 to 15 seconds so that the lash adhesive liner formulation becomes tacky prior to pressing the lash band against the tacky lash adhesive layer. In an additional option, an additional amount of the lash adhesive liner formulation may be applied to a corner of the lash band after the lash band has been pressed into position.

In some embodiments, the method may include applying the lash adhesive liner formulation to the eyelid using a pen. Depending upon how long it has been since the lash adhesive liner pen was last used, the lash adhesive liner pen may be shaken prior to applying the layer of lash adhesive liner formulation to the eyelid. Shaking the pen may mix the components of the lash adhesive liner formulation within the pen prior to each use.

Some embodiments provide a method of removing the lash from the eyelid that may prolong the useful life of the lash. Because the lash adhesive liner formulation has a unique combination holding strength and clean release, a given lash may be worn about 30 times with proper care. The method may include gently removing the lash by lifting an end of the band of the lash off of the skin at the outer corner of the eye, then gently lifting the end of the band to gently remove the remainder of the lash. It is recommended to never pull on the hair or filaments of a lash during removal, but rather to always use the band. Removing the lash in this manner will leave most of the lash adhesive liner formulation on the eyelid, such that the lash is clean and undamaged. It is a significant advantage and benefit that the present lash adhesive liner formulation leaves the lash clean and undamaged, since any adhesive or glue residue that is left on the lash after removal of the lash may damage or mangle the lash, may pull on the person's natural eyelashes, and/or may require time and effort to remove the adhesive or glue reside from the lash before storage and/or subsequent use. Not only is the lash adhesive liner formulation advantageous for leaving the lash clean and undamaged, but the lash adhesive liner formulation also releases well from the eyelid where the lash adhesive liner formulation was applied.

### Definitions

An “eyelash” is one of the natural hairs that grows along an edge of an eyelid of a person.

The term “eyelashes” is the plural form of “eyelash” and typically refers to all of the natural hairs that grow along an edge of one or both eyelids of a person.

A “lash” refers to a single instance or unit of false eyelashes that includes an arrangement of hairs or synthetic filaments secured together with a single band by which the “lash” may be secured to one eyelid of a person for cosmetic purposes.

A “lash band” refers a strip of material, such as a plastic, that secures a plurality of individual hairs or synthetic filaments together in a fixed arrangement as a single unit to form a “lash.”

A “pair of lashes” refers to a pair of false eyelashes (i.e., two “lashes”), where each instance of the false eyelashes (i.e., each “lash”) may be secured to an eyelid of a person for cosmetic purposes.

An “eyelash extension” is a single hair or synthetic filament that can be adhered to a single eyelash with eyelash glue.

A “lash adhesive” is a substance that is used to temporarily secure a “lash” (in particular, the “lash band”) to an eyelid of a person.

“Eyeliner” (or simply, “liner”) is a type of cosmetic applied around the contours of the eyes.

### EXAMPLES

In some embodiments, a lash adhesive liner formulation is provided in clear, black, and brown colors. For each of the clear, black and brown colors of lash adhesive liner formulation, the concentration of each ingredient in weight percent of the total lash adhesive liner formulation is provided in the table below:

| INCI name | CAS No. | Clear | Black | Brown |
|---|---|---|---|---|
| Styrene acrylate copolymer | 9010-92-8 | 35 | 13.2 | 45 |
| ACS Reagent Grade Water | 7732-18-5 | 50.4 | 32 | 32 |
| 1,2-Propanediol | 57-55-6 | 10 | 7.3 | 7.3 |
| Polyvinyl alcohol | 25213-24-5 | 2 | 1 | 1 |
| Polysorbate 80 | 9005-65-6 | 2 | 0.9 | 0.9 |
| Phenoxyethanol | 122-99-6 | 0.27 | 0.27 | 0.27 |
| Octanediol | 1117-86-8 | 0.21 | 0.21 | 0.21 |
| Ethylhexyl glycerin | 70445-33-9 | 0.12 | 0.12 | 0.12 |
| CI 77266 (Carbon black; | 1333-86-4 | — | 45 | — |
| black colorant) | | | | |
| CI 77499 (triiron tetraoxide; | 12227-89-3 | — | — | 1.2 |
| black colorant) | | | | |
| CI 77491 (diiron trioxide; | 1309-37-1 | — | — | 6 |
| red colorant) | | | | |
| CI 77492 (iron oxide; | 20344-49-4 | — | — | 6 |
| yellow colorant) | | | | |

The lash adhesive liner formulations set out in the above table include only nontoxic materials, such that there are no ingredients that can irritate the sensitive eye area. The lash adhesive liner formulations are vegan, cruelty-free, windproof and do not contain any latex. Furthermore, the lash adhesive liner formulation won't damage the wearer's natural lashes.

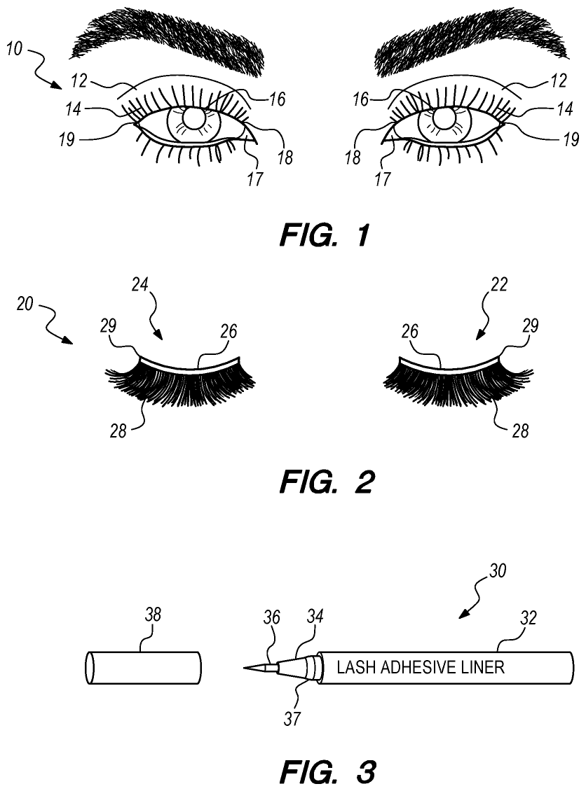

FIG. 1 is a diagram of a person's eyes 10, upper eyelids 12, and natural eyelashes 14. The natural eyelashes 14 grow at the lower edge or “lashline” 16 of each eyelid 12 from an inner corner 18 near the tear duct 17 to an outer corner 19 opposite the tear duct. The length and fullness of natural eyelashes 14 will vary from person to person. Depending upon a desired appearance, the person may desire to enhance the appearance of their eyelashes. One manner of achieving that enhanced appearance is to wear false eyelashes (i.e., “lashes”).

FIG. 2 is a diagram of a pair of lashes 20 including a left lash 22 and a right lash 24 (from the perspective of the person that will wear the lashes). Each lash 22, 24 includes a lash band 26 than secures an end of each hair or filament 28 that is included in the lash. The last band 26 has been illustrated as a clear band in order to delineate the lash band from the hairs or filaments 28 that are secured to the lash band. However, it should be recognized that the lash band may be any color.

The number, length and materials of the hairs or filaments 28 may vary from one pair of lashes to another. Furthermore, a given lash style may, for example, have a particular combination of lash volume, lash shape, lash color and/or lash band color. In several non-limiting examples, a lash volume may be natural, medium, or maximum; a lash shape may be flared or round; a lash color may be black or brown; and a lash band color may be clear, black, or brown. Non-limiting examples of lash materials include human hair; animal fur such as mink, sable or fox; and/or synthetic lashes such as acrylic resin or polybutylene terephthalate (PBT).

The lash band 26 (sometimes also referred to as a “lash strip”) may be translucent or opaque. An opaque lash band may be any desired color, such as black or brown. A translucent or clear lash band may less detectable, depending upon the person's use of other cosmetics on the eyelid. Furthermore, the lash band may be a full strip lash band that extends across the entire edge of the eyelid adjacent and above the natural eyelashes. In reference to both FIGS. 1 and 2, a full strip lash band 26 may extend along the upper lashline from an inner corner 18 of the eye 10 near or adjacent the tear duct 17 to an outer corner 19 (or “outer V”) of the eye 10. Typically, even a full strip lash band 26 will be trimmed to extend from a first eyelash 14 that is nearest to the inner corner 18 to a last eyelash 14 that is nearest to the outer corner 19. Alternatively, the lash band may be a partial strip lash band, such as a ¾ lash band or a small cluster. Furthermore, the lash band may have already been custom trimmed by the person during a previous wearing of the lashes. When trimming lashes, care should be taken to trim the lash band 26 from the outer corner or end 29 of the band that is furthest away from the eye duct.

FIG. 3 is a diagram of a pen 30 for applying a lash adhesive liner formulation. The pen 30 includes a cylindrical chamber 32 that establishes an internal fluid reservoir where the lash adhesive liner formulation is contained. The cylindrical chamber 32 narrows at a distal end 34 to secure a tip 36 made of pressed fibers, such as pony hair, or a porous material, such as felt. The distal end 34 may also form a feature 37, such as circumferential ridges, that cooperate with internal ridges (not shown) of a removable cap 38 to secure the cap over the distal end 34 when the pen 30 is not in use. The lash adhesive liner formulation within the chamber 32 is wicked through the porous tip 36 for controlled delivery onto a surface that touches the porous tip 36.

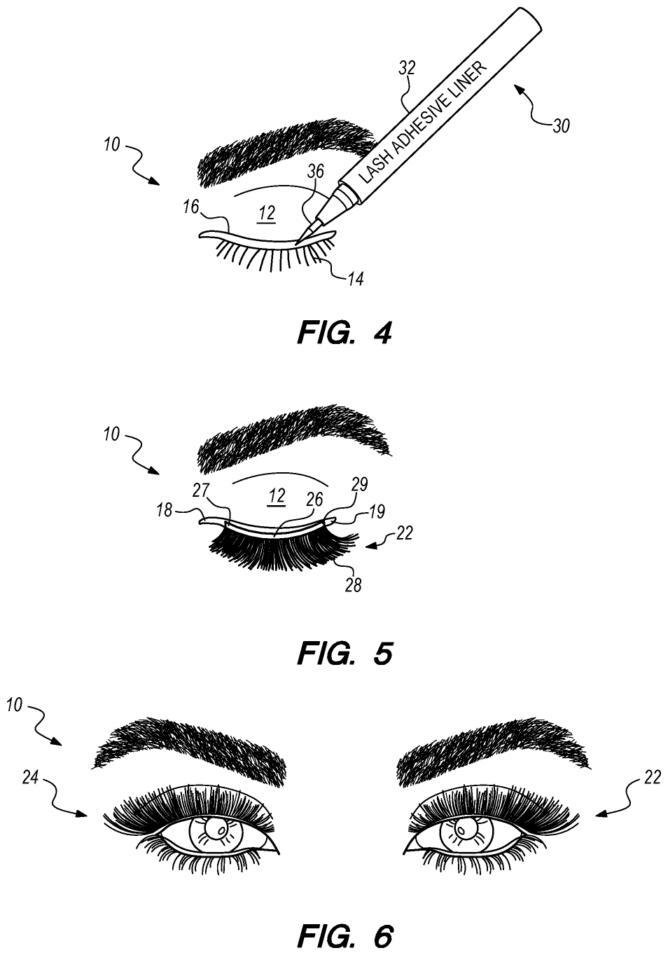

FIG. 4 is a diagram of the pen 30 being used to apply a lash adhesive liner formulation on the eyelid 12 of the person's left eye 10 adjacent the eyelashes 14. The person holds their eye closed so that their natural eyelashes 14 are generally downwardly directed and the skin of the eyelid 12 directly above lashline 16 is easily accessible to the tip 36 of the pen 30. By moving the tip 36 of the pen 30 along the lashline 16, the person or their assistant may cause the lash adhesive liner formulation in the cylindrical chamber 32 to be controllably delivered to the lashline 16. The width of an area of the eyelid 12 that receives the lash adhesive liner formulation may be controlled by the angle of the pen 30 relative to the eyelid 12, the amount of pressure applied by the tip 36 to the eyelid 12, and by moving the placement of the pen over multiple strokes of the pen 30. Furthermore, a thicker coating or film of the lash adhesive liner formulation may be applied by reducing the speed at which the tip 36 is moved across the skin and/or making multiple passes of the tip 36 over the same area so as to apply multiple layers of the lash adhesive liner formulation.

The lash adhesive liner formulation may be applied to the skin of the eyelid 12 in any known manner. For example, the lash adhesive liner formulation can be applied directly to a bare eyelid or over eyeshadow that has already been applied to the eyelid. In some embodiments, the lash adhesive liner formulation may be applied to the eyelid with a standard eyeliner pen, such as a precision eyeliner pen. When an eyeliner pen is used to apply the lash adhesive liner formulation, the eyeliner pen should be stored with the adhesive eyeliner tip upside down (i.e., with the tip pointed downward) for optimal flow of the lash adhesive liner formulation to the tip.

In one method, the lash adhesive liner pen 30 is shaken well prior to application of the lash adhesive liner formulation to the eyelid 12. Then, two to three generous layers of lash adhesive liner formulation may be applied just above the lashline 16 of a desired eyelid. Optionally, an additional amount of the lash adhesive liner formulation may be applied to the back side of the lash band 26 (see FIG. 2) itself for extra hold. The lash adhesive liner formulation should then be allowed to dry in place for approximately 5 to 15 seconds or until the lash adhesive liner formulation becomes tacky to the touch.

FIG. 5 is a diagram of the same left eye 10 shown in FIG. 4 after pressing a band 26 of the left lash 20 down onto the lash adhesive liner formulation that was applied just above (immediately adjacent) the lashline 16 in FIG. 4. After allowing about 5 to 15 seconds for the lash adhesive liner formulation to become tacky, the eyelash band was pressed against the eyelid in the area above the lashline 16 where the lash adhesive liner formulation was applied. Accordingly, the lash adhesive liner formulation is placed under pressure and will bond with the eyelash band 26. This same procedure or a similar procedure should be followed for the other eye 10 of the person (see FIG. 1).

In one option, the lash 20 may be pressed against the lash adhesive liner formulation using an applicator (not shown). An applicator may resemble a pair of slant tweezers, but may feature a wider tip with more surface area to grip onto the entire length of a lash band letting you position and place the lash band flatly along the lashline 16 of the eyelid 12. In another option, the lash adhesive liner formulation may be further applied to an inner corner of the lash 20 that is already in place on the eyelid. This further application of the lash adhesive liner formulation may improve the hold and prevent the lash band 26 from lifting away from the eyelid 12.

FIG. 6 is a diagram of the person's eyes 10 after securing the left and right lashes 22, 24 to the left and right eyes, respectively. The lashes 22, 24 provide greater length and volume than the natural lashes 14 alone (see FIG. 1). Furthermore, the lash adhesive liner formulation may contain one or more colorant such that the formulation may substitute for eyeliner. Optionally, a matching eyeliner may be used to supplement the lash adhesive liner formulation, such as if the person wants to extend the eyeliner well beyond the lashline.

Referring back to FIG. 5, the person may remove the lash 22 from the eyelid 12, either to reposition the lash, replace the lash with a different lash, or leave the lash off. Regardless of the purpose, the person may grab the inner end 27 of the lash band 26 (i.e., the end of the lash band nearest the inner corner 18 of the eye 10) and gently lift the end 27 of the lash band away from the eyelid 12. It is recommended to never pull on the hair or filaments of a lash during removal, but rather to always use the band. Removing the lash in this manner will leave most of the lash adhesive liner formulation on the eyelid, such that the lash is clean and undamaged.

Since the lash adhesive liner formulation releases the lash band 26 cleanly, the lash 22 or another lash may be immediately be reapplied to the eyelid. In addition, the improved release of the lash adhesive liner formulation relative to a typical lash glue causes there to be reduced levels of stress on the lash band 26 as it is being pulled during removal. Furthermore, the improved release also means that there is little or no lash adhesive liner formulation that remains on the lash band 26 that was just removed. Relative to a typical lash glue, the lash adhesive liner formulation will enable the lash to last longer. Because the lash adhesive liner formulation has a unique combination holding strength and clean release, a given lash may be worn about 30 times with proper care.

The terminology used herein is for the purpose of describing particular embodiments only and is not intended to limit the scope of the claims. As used herein, the term “about” may be used synonymously with “approximately” to mean that an amount is not exact and that some variation is contemplated, perhaps due to measurement error or unintentional variations in the concentration of sourced components. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the embodiment.

The corresponding structures, materials, acts, and equivalents of all means or steps plus function elements in the claims below are intended to include any structure, material, or act for performing the function in combination with other claimed elements as specifically claimed. Embodiments have been presented for purposes of illustration and description, but it is not intended to be exhaustive or limited to the embodiments in the form disclosed. Many modifications and variations will be apparent to those of ordinary skill in the art after reading this disclosure. The disclosed embodiments were chosen and described as non-limiting examples to enable others of ordinary skill in the art to understand these embodiments and other embodiments involving modifications suited to a particular implementation.

What is claimed is:

1. A lash adhesive liner formulation, comprising:
a pressure-sensitive adhesive that is activated by pressing a lash band against the pressure-sensitive adhesive that has been applied to the eyelid; and
between about 0.02 and about 0.3 weight percent glycerin.

2. The lash adhesive liner formulation of claim 1, wherein the glycerin concentration is between about 0.12 weight percent and about 0.3 percent.

3. The lash adhesive liner formulation of claim 1, wherein the amount of glycerin in the formulation is effective to provide skin moisturization and improve the release of the lash adhesive liner formulation from the skin without causing the lash adhesive liner formulation to be slick to the touch.

4. The lash adhesive liner formulation of claim 1, further comprising:
one or more colorant compound that establishes a color of the lash adhesive liner formulation, wherein the lash adhesive liner formulation serves as a substitute for eyeliner.

5. The lash adhesive liner formulation of claim 1, wherein the pressure-sensitive adhesive enables a lash band to be both removable after being applied to the pressure-sensitive adhesive and re-stickable after being removed from the pressure-sensitive adhesive.

6. The lash adhesive liner formulation of claim 1, characterized in that the lash adhesive liner formulation remains on the eyelid as a lash band is removed.

7. The lash adhesive liner formulation of claim 1, characterized in that two to three layers of the lash adhesive liner formulation applied to the skin with a tip of an eyeliner pen provides a coating having sufficient thickness to secure a lash band that is pressed against the layers.

8. The lash adhesive liner formulation of claim 1, wherein the pressure-sensitive adhesive includes from 1 to 2 weight percent polyvinyl alcohol.

9. The lash adhesive liner formulation of claim 8, further comprising:
from 13 to 45 weight percent styrene acylate copolymer.

10. A lash adhesive liner formulation, comprising:
about 35 weight percent styrene acrylate copolymer;
about 50 weight percent water;
about 10 weight percent 1,2-propanediol;
about 2 weight percent polyvinyl alcohol;
about 2 weight percent polysorbate 80;
about 0.27 weight percent phenoxyethanol;
about 0.21 weight percent octanediol; and
about 0.12 weight percent ethylhexylglycerin.

11. The lash adhesive liner formulation of claim 10, characterized in that the lash adhesive liner formulation may be applied to the skin through a tip of an eyeliner pen.

12. The lash adhesive liner formulation of claim 10, characterized in that two to three layers of the lash adhesive liner formulation applied to the skin with a tip of an eyeliner pen provides a coating having sufficient thickness to secure a lash band that is pressed against the layers.

13. A lash adhesive liner formulation, comprising:
between about 13 and about 45 weight percent styrene acrylate copolymer;
about 32 weight percent water;
about 7.3 weight percent 1,2-propanediol;
about 1 weight percent polyvinyl alcohol;
about 0.9 weight percent polysorbate 80;
about 0.27 weight percent phenoxyethanol;
about 0.21 weight percent octanediol;
about 0.12 weight percent ethylhexylglycerin; and
one or more colorant composition.

14. The lash adhesive liner formulation of claim 13, wherein the styrene acrylate copolymer has a concentration of about 13 weight percent, and wherein the one or more colorant includes about 45 weight percent carbon black.

15. The lash adhesive liner formulation of claim 13, wherein the styrene acrylate copolymer has a concentration of about 45 weight percent, and wherein the one or more colorant includes about 1.2 weight percent triiron tetraoxide, about 6 weight percent diiron trioxide, and about 6 weight percent iron oxide.

16. The lash adhesive liner formulation of claim 13, characterized in that the lash adhesive liner formulation becomes tacky in about 5-15 seconds after being applying to an eyelid.

17. A method of securing a lash to a lashline of an eyelid, comprising:
applying a layer of a lash adhesive liner formulation to an eyelid, wherein the lash adhesive liner formulation comprises a pressure-sensitive adhesive and greater than 0.2 weight percent glycerin; then
allowing the lash adhesive liner formulation that has been applied to the eyelid to dry for 5-15 seconds so that the lash adhesive liner formulation becomes tacky; and then
pressing a lash band of a lash firmly against the tacky lash adhesive liner formulation.

18. The method of claim 17, further comprising:
applying an additional amount of the lash adhesive eyeliner to the lash band; and then
allowing the lash adhesive liner formulation that has been applied to the lash band to dry for 5-15 second so that the lash adhesive liner formulation becomes tacky prior to pressing the lash band against the tacky lash adhesive layer.

19. The method of claim 17, wherein the lash adhesive liner formulation is applied to the eyelid using an eyeliner pen.

20. The method of claim 19, where the step of applying a layer of lash adhesive liner formulation to an eyelid includes applying two to three layers of the lash adhesive liner formulation to the eyelid.
